(19) United States
(12) Patent Application Publication
(10) Pub. No.: US 2009/0082344 A1
(43) Pub. Date: Mar. 26, 2009

(54) Methods and systems for treating disease

(75) Inventors: Edward K.Y. Jung, Bellevue, WA (US); Lowell L. Wood, JR., Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, Bellevue, WA (US)

(21) Appl. No.: 12/283,158

(22) Filed: Sep. 9, 2008

Related U.S. Application Data
(62) Division of application No. 11/525,760, filed on Sep. 22, 2006.
Application No. 11/525,760 is a continuation-in-part of application No. 11/487,180, filed on Jul. 13, 2006.
Application No. 11/487,180 is a continuation-in-part of application No. 11/487,133, filed on Jul. 13, 2006.

(51) Int. Cl. A61K 31/536; G06F 19/00

(52) U.S. Cl. 514/230.5; 702/19

(57) ABSTRACT

Methods and systems described herein are applicable to the identification of pathogens, pathogenic variants and applicable treatments or remedies. In some embodiments, the pathogen or pathogens bears a causal relationship to a disease state.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. [USAN to be assigned by PTO], entitled Methods and Systems for Molecular Inhibition, naming Edward K.Y. Jung, Nathan P. Myhrvold and Lowell L. Wood, Jr. as inventors, filed contemporaneously herewith, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present applicant entity has provided above a specific reference to the application(s) from which priority is being claimed as recited by statute. Applicant entity understands that the statute is unambiguous in its specific reference language and does not require either a serial number or any characterization, such as “continuation” or “continuation-in-part,” for claiming priority to U.S. patent applications. Notwithstanding the foregoing, applicant entity understands that the USPTO's computer programs have certain data entry requirements, and hence applicant entity is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### SUMMARY

Methods and systems described herein involve identifying primary pathogens as well as variants of the pathogens and treatments. In some embodiments, pathogens are causally linked to a disease state. Also included are systems containing instructions for identification of pathogens and pathogen variants as well as those for identifying treatments for the pathogens.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 outlines method steps carried out in some embodiments discussed herein.

Shown in FIG. 2 are method steps from further embodiments.

FIG. 3 describes method steps applicable in still further embodiments.

FIG. 4 describes sets of instructions from computer programs described herein.

FIG. 5 outlines sets of instructions from computer programs included in some embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and/or firmware.

In one aspect, methods described herein include identifying a primary pathogen, identifying a primary treatment targeting the primary pathogen, predicting a first pathogenic variant of the primary pathogen, identifying a set of first variant treatments targeting the first pathogenic variant, predicting a second pathogenic variant of the primary pathogen and identifying a set of second variant treatments targeting the second pathogenic variant. Also described herein are methods that include identifying a pathogen that is a primary pathogenic cause of a disease state, identifying at least one variant of the pathogen and identifying at least one treatment targeted against each identified variant of the pathogen. Methods described herein also include identifying a primary pathogen causally linked to a disease state, identifying a treatment for the disease state focused against the primary pathogen, identifying a secondary pathogen causally linked to the disease state and identifying at least one treatment for the disease state targeted against the secondary pathogen.

Also described herein are systems including computer programs for use with a computer system and wherein the computer program includes a plurality of instructions including but not limited to a first set of instructions for identifying at least one primary pathogen associated with a given disease state, a second set of instructions for predicting at least one pathogenic variant of a primary pathogen and a third set of instructions for identification of at least one second treatment targeting at least one predicted pathogenic variant. Systems described herein also include those comprising a computer readable medium including a computer program for use with a computer system, said computer program having a plurality of instructions including a set of instructions to identify a primary pathogen, a set of instructions to identify a remedy for the primary pathogen, a set of instructions to identify at least one additional pathogen and a set of instructions to identify at least one remedy for at least one additional pathogen.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

Methods and systems described herein include the identification of pathogens. As used herein, “pathogen” refers to at least one agent that disrupts the normal metabolic state of an organism, including but not limited to causing symptomatic disease. Similarly, as used herein “pathogenic” refers to at least one agent with qualities of a pathogen. As used herein, “primary” refers to the first identified entity of that type and, similarly, “secondary” refers to the second identified entity of that type. For example, a primary pathogen is the first pathogen identified as part of a method or system and a second pathogen identified would be a “secondary” pathogen. In some embodiments, there are “additional pathogen(s)” which herein refers to pathogen(s) identified after the primary pathogen. A pathogen may be associated with a disease state. As used herein, “disease state” includes clinically diagnosed disease as well as disruptions in the normal metabolic state of an organism that have not been diagnosed as clinical disease. For example, in some infections there is an extensive delay between the time of infection and clinically evident symptoms, including but not limited to the often extensive delay between HIV infection of an individual and onset of AIDS symptoms. Other examples of a disease state that may not be diagnosed as clinical disease include being a chronic carrier of hepatitis B or C viruses (HBV or HCV), or asymptomatic infection with Helicobacter pylori, chlamydia, gonorrhea or human papillomavirus (HPV). In some embodiments, methods may include identifying an additional pathogen causally linked to the disease state or identifying a plurality of additional pathogens causally linked to the disease state. For example, there may be multiple types of pathogens causally linked to a disease state or multiple variants of one type of pathogen causally linked to a disease state. Examples of multiple types of pathogen causally linked to a disease state include hepatitis A, B, C, D and E viruses, which are different viruses with distinct etiology but are all individually linked to inflammation of the liver. Some examples of multiple variants of one type of pathogen include the extensive strain variation in Escherichia coli and HIV. More information on HIV strain evolution may be found in Rambaut et al., “The Causes and Consequences of HIV Evolution”, Nature Reviews Genetics 5:52-61 (2004), which is herein incorporated by reference. In some embodiments, there may be a “pathogenic cause of the disease state”, which herein refers to a pathogen or pathogens which are necessary for the onset of the disease state. Although a pathogen or pathogens may be a pathogenic cause of the disease state, in some embodiments other factors or events may be required for development of disease. For example, the hepatitis D virus is unable to replicate in humans in the absence of the hepatitis B virus, and therefore infection with hepatitis B is required for infection with hepatitis D to occur and hepatitis D-based disease to develop. More details regarding hepatitis D is available in the “Hepatitis Delta” report from the World Health Organization (WHO) 2001, which is herein incorporated by reference. In various embodiments, the disease state may affect a domestic animal, a non-domesticated animal and/or a human. In some embodiments, the disruption of the normal metabolic state of an organism is a pre-disease state that is likely to progress to disease if untreated, but in others there is a disruption of the normal metabolic state but the condition is unlikely to progress to clinically evident disease. For example, in some locales Helicobacter pylori infections are endemic and yet only a subset of infected individuals develop H. pylori-related ulcers and other disease. More information regarding H. pylori population rates of infection and development of related diseases is available in Danesh et al., “Chronic Infection with Helicobacter pylori, Chlamydia pneumoniae, or Cytomegalovirus; Population Based Study of Coronary Heart Disease”, Heart 81:245-247 (1999), which is herein incorporated by reference. In some embodiments, the pathogen is an agent that is widely known to be associated with disease while in others it is an agent that is not widely identified as being associated with disease. In some embodiments, the pathogen and/or at least one pathogenic variant is a virus, a bacterium, a yeast, a mold, a fungus, a mycoplasma, a ureaplasma, a chlamydia, a rickettsia, a nanobacterium, a prion, an agent responsible for a transmissible spongiform encephalopathy (TSE), a multicellular parasite, a protein, an infectious protein, a nucleic acid, a metabolic by-product, a cellular by-product, or a toxin. As used herein, “identification” refers to the determination that an agent is acting as described in that particular context. For example, the identification of a pathogen would generally include the determination that the agent is disrupting the normal metabolic state of an organism. In some embodiments, identification of the primary pathogenic cause of the disease state includes accessing information from a database.

In some embodiments, methods and systems described herein include estimating the probability of existence of a pathogen. Some embodiments of methods and systems described herein include a set of instructions for estimating the probability that at least one identified pathogen exists. As used herein, “estimating the probability of existence” refers to making estimates of the probability of existence of an entity of that type in a given circumstance. Estimating the probability of existence includes but is not limited to making statistical probability estimates based on models of disease incidence or infection rates as well as estimating the probability based on direct observation of a given situation or circumstance. Estimating the probability of existence includes, but is not limited to, clinical differential diagnosis techniques. As a non-limiting example of estimating the probability of existence of different pathogens associated with rhinosinusitis in a clinical setting, see Schied and Hamm, American Family Physician 70:1685-1692 (2004), which is herein incorporated by reference. In some embodiments, estimating the probability of existence includes the use of computing devices. As an example of the use of computing devices in estimating the probability of existence of pathogens in a clinical setting, see Burdette et al., Annals of Clinical Microbiology and Antimicrobials 3:22 (2004), which is herein incorporated by reference. Some methods described herein include identifying an additional pathogen causally linked to the disease state and estimating the probability of existence of the additional pathogen in a given individual. In some embodiments, the steps of identifying an additional pathogen causally linked to the disease state and estimating the probability of existence of the additional pathogen in a given individual are repeated. In some embodiments, the steps are repeated until there is less than about a 1% probability and/or less than about a 5% probability association between the identified additional pathogen and a given individual. In embodiments that include a set of instructions for estimating the probability that any identified additional pathogen exists, the estimated probability may be greater than about 5%, greater than about 1% and/or greater than about 0.1%.

As described herein, “variant” refers to at least one entity that is distinct from, but identifiably similar to, at least one other entity of the same general type. For example, a pathogenic variant is a variant form of the same type of pathogen otherwise identified in the method or system. As is well known to those of skill in the art, pathogenic variants may arise due to genetic mutation, which may be of any type including nucleotide substitutions, insertions, deletions, inversions and a combination of mutation types. More information regarding the rates of genetic mutation in some pathogens is available in Awadalla, “The Evolutionary Genomics of Pathogen Recombination”, Nature Reviews Genetics 4:50-60 (2003), which is herein incorporated by reference. Variants may be present at the onset of the method or system and they also may arise during the operational course of the method or system. As an example of variants that could be present at the onset of a method or system, there is at least one penicillin-resistant variant of Treponema pallidum causing syphilis circulating worldwide that was not identified until after several individuals failed conventional antibiotic treatment (see Lukehart et al., “Macrolide Resistance in Treponema pallidum in the United States and Ireland” New England Journal of Medicine 351(2):154-158 (2004), which is herein incorporated by reference). As an example of variants arising during the operational course of a method or system, it has been recognized that the HIV virus mutates extremely quickly during the course of infection of an individual, including during treatment (see Coffin, “HIV Population Dynamics in Vivo: Implications for Genetic Variation, Pathogenesis and Therapy” Science 267: 483-489 (1995), and Ribero and Bolnhoeffer, PNAS 97(14): 7681-7686 (2000), which are herein incorporated by reference). In some embodiments, the secondary pathogen and/or additional pathogen(s) are a variant form of the primary pathogen. In some embodiments, methods include predicting at least one pathogenic variant. In some embodiments, a plurality of pathogenic variants are predicted. As used herein, “predicting” refers to foretelling by any means, including on the basis of observation, experience, or scientific reason. By way of example, for some pathogens an extensive history of pathogen variant evolution has been described which yields insight into future likely variants, as is described for influenza A in Smith et al., “Mapping the Antigenic and Genetic Evolution of Influenza Virus” Science 305 (5682) 371-376 (2004) and Both et al., “Antigenic Drift in Influenza Virus H3 Hemagglutinin from 1968 to 1980: Multiple Evolutionary Pathways and Sequential Amino Acid Changes at Key Antigenic Sites” Journal of Virology 48(1):52-60 (1983), which are herein incorporated by reference. As is known to those of skill in the art, in some embodiments all possible variants are predicted to occur while in others only a few may be likely. For more information regarding the evolutionary adaptation of microorganisms, see Elena and Lenski, “Evolution Experiments with Microorganisms: the Dynamics and Genetic Bases of Adaptation” Nature Reviews Genetics 4:457-469 (2003), which is herein incorporated by reference. In some embodiments, the predicted pathogenic variant is a previously identified variant. In some embodiments, predicting at least one pathogenic variant is in response to a molecular model of the primary pathogen. A molecular model refers to a structural model of a particular molecule or class of molecules and may include a molecule or molecules in their entirety or it may include only a portion of a molecule or molecules. As used herein a molecular model includes, but is not limited to, chemical, atomic and physical models, which may include tertiary structure including one or more atomic coordinates, linear diagrams, space-filling structures or predictions, geometric predictions, structures based on functional groups or structures based on chemical or molecular bonds. The molecular models contemplated herein may or may not be visually presented and may or may not be represented in a physical form. In some embodiments, the prediction of at least one pathogenic variant includes accessing information from a database.

Methods and systems described herein include identifying treatments. As used herein, “treatment(s)” refers to remedy(s) which may be administered or applied to alleviate the disruption in normal metabolic state caused by a pathogen or pathogens. In some embodiments, treatments may be chosen to alleviate disease symptoms or they may be chosen to alleviate a pre-disease state. As used herein, “first treatment(s)” are those designed to alleviate disruptions in normal metabolic states due to primary pathogens while “second treatment(s)” are those designed to alleviate disruptions in normal metabolic states due to secondary pathogens. In some embodiments, a plurality of second treatments are identified. In some embodiments, treatments are targeted against each identified variant of the pathogen. As used herein, “targeted against” refers to at least one treatment that is applicable to a given pathogen. Systems as described herein include a set of instructions for identification of at least one first treatment targeting the primary pathogen responsible for a given disease state. Some methods include selecting at least one treatment. As used herein, “selecting” refers to choosing at least one from a set or group. Some methods include selecting at least one identified first variant treatment and selecting at least one identified second variant treatment. Some methods include administering the selected first variant treatment and the selected second variant treatment. Methods also include treating the disease using a combination of treatments, including at least one selected first treatment and at least one selected second treatment. Multiple treatments may be applied at the same time or they may be applied at different times. Multiple treatments may have the same route of delivery into the body of the individual being treated. Treatments may be administered by any means known to those of skill in the art, including orally, via injection, via intravenous administration, transdermally, nasally, through the lung tissue or via inhalation. In embodiments where there are multiple treatments, at least one may be administered at a single time, or at least one may comprise a series of treatments carried out over time. In some embodiments, selecting at least one treatment includes accessing information from a database. In some embodiments, selecting at least one treatment is made in response to a molecular model of a primary pathogen and/or a pathogenic variant. For example, the response of different variants of hepatitis B (HBV) to a range of treatments has been described by Ono et al., “The Polymerase L528M Mutation Cooperates with Nucleotide Binding-site Mutations, Increasing Hepatitis B Virus Replication and Drug Resistance”, Journal of Clinical Investigation 107(4):449-455 (2001), which is herein incorporated by reference. In some embodiments, the remedy for an additional pathogen is a variant of the remedy for the primary pathogen. Some systems as disclosed herein include a set of instructions for selecting a combination of at least one remedy for the primary pathogen and at least one remedy for an additional pathogen.

In some embodiments of the methods described herein, the identification of the primary pathogenic cause of the disease state is made in response to the results of biochemical testing. In some embodiments, identifying the at least one variant of the pathogen includes detecting a variant through biochemical testing. As used herein, “biochemical testing” refers to testing that involves biochemical assays or biomolecules. By way of non-limiting examples, biochemical testing includes routine biomedical testing procedures including pathological tests, endocrine testing, enzymatic tests, immunological testing, toxicology tests and pharmacological tests. As used herein, “result” refers to a consequence, conclusion or data obtained. In some embodiments, the identification of the primary pathogenic cause of the disease state is made in response to the results of examination of tissue. As used herein, “examination of tissue” refers to tissues of an organism affected by the pathogen, including pathological examination, routine clinical examination and examination at autopsy. In some embodiments, identifying the at least one variant of the pathogen includes detecting a variant through physical means. As used herein, “detecting by physical means” refers to any type of physical detection means, including but not limited to optical, sonic, radiological, olfactory or tactile means. In some embodiments, identifying the at least one variant of the pathogen includes referencing a database of predicted variants. In some embodiments, the database of predicted variants includes a database of predicted DNA variants of the pathogen. In some embodiments, the database of predicted variants includes a database of predicted protein variants of the pathogen. In some embodiments, identifying the at least one variant of the pathogen includes molecular modeling. As used herein, molecular modeling is the creation of or reference to a molecular model. In some embodiments, the modeling comprises modeling based on the route of infection of at least one variant of the pathogen. This may include modeling of specific cell surface receptors, cellular pores or cell membrane components.

Some embodiments of the methods described herein include identifying a treatment targeted against the primary pathogenic cause of the disease. Methods as described herein may also include selecting at least one treatment targeted against the primary pathogenic cause of the disease as well as selecting at least one treatment targeted against at least one variant of the pathogen. Treatments may be further selected to be compatible for co-delivery. As used herein, “co-delivery” refers to treatment delivery that occurs at the same or similar time, and/or that is administered by the same or similar method. Some embodiments include selecting at least one treatment targeted against at least one variant of the pathogen. Some embodiments include identifying at least one additional variant of the pathogen and identifying at least one additional treatment targeted against each additional variant of the pathogen. These additional variant(s) and treatment(s) may be identified at the same time as each other or in series. Depending on the embodiment, there may be a few additional variant(s) with a corresponding few additional treatment(s) or there may be a large number of both variants and corresponding treatments.

Some methods and systems described herein include a set of instructions for predicting the response of the primary pathogen to at least one first treatment. As used herein “response” refers to the activity or inhibition of previous activity resulting from the treatment. Methods and systems described herein also include a set of instructions for predicting the response of at least one pathogenic variant to at least one first treatment. Some embodiments include a set of instructions for predicting the response of at least one pathogenic variant to the second treatment. Systems also include those comprising a set of instructions for predicting the response of the combination of the primary pathogen and at least one pathogenic variant to the combination of at least one first and at least one additional treatment. Some embodiments include a set of instructions that refer to a database. The database may comprise known pathogens, known disease states, known variants or predicted mutations of pathogens, molecular structures of pathogens or a combination of at least two of these. In some embodiments, a plurality of pathogenic variants and/or a plurality of second treatments are identified. Some embodiments comprise a set of instructions for designating a grouping of pathogenic variants. The set of instructions for designating a grouping of pathogenic variants may include at least one user chosen parameter. At least one user chosen parameter may include at least one of the following; geographic location, a temporal variable, the route of transmission of the pathogen, the species of the individual or individuals affected by the disease state, the gender of the individual or individuals affected by the disease state, the age of the individual or individuals affected by the disease state and/or the results of laboratory testing involving the pathogen.

Some methods and systems described herein comprise a set of instructions including referencing a database of information regarding previously identified pathogens. By way of example, types of information that might be included in such a database, depending on the embodiment, would include pathogen name(s), molecular structure, pathogenic classification, modes of infection and known treatment options.

In some embodiments that include sets of instructions including those to identify a secondary pathogen, to identify at least one remedy for the secondary pathogen and for estimating the probability that any identified secondary pathogen exists, these steps may be repeated until the probability of existence of the majority of additional secondary pathogens identified is lower than a set target level. In some embodiments, the set target level is lower than about 0.01%, lower than about 0.1%, lower than about 1% and/or lower than about 5%.

Systems described herein include computer readable media containing instructions, which, when run on a computer cause the computer to perform the steps of identifying a primary pathogen, identifying a primary treatment targeting the primary pathogen, predicting a first pathogenic variant of the primary pathogen, identifying a set of first variant treatments targeting the first pathogenic variant, predicting a second pathogenic variant of the primary pathogen and identifying a set of second variant treatments targeting the second pathogenic variant. Systems described herein also include computer readable media containing instructions, which, when run on a computer cause the computer to perform the steps of identifying a primary pathogen causally linked to a disease state, identifying a treatment for the disease state focused against the primary pathogen, identifying a secondary pathogen causally linked to the disease state and identifying at least one treatment for the disease state targeted against the secondary pathogen. Systems described herein also include computer readable media containing instructions, which, when run on a computer cause the computer to perform the steps of identifying an additional pathogen causally linked to the disease state and estimating the probability of existence of the additional pathogen in a given individual.

The Figures further describe non-limiting aspects of the methods and systems.

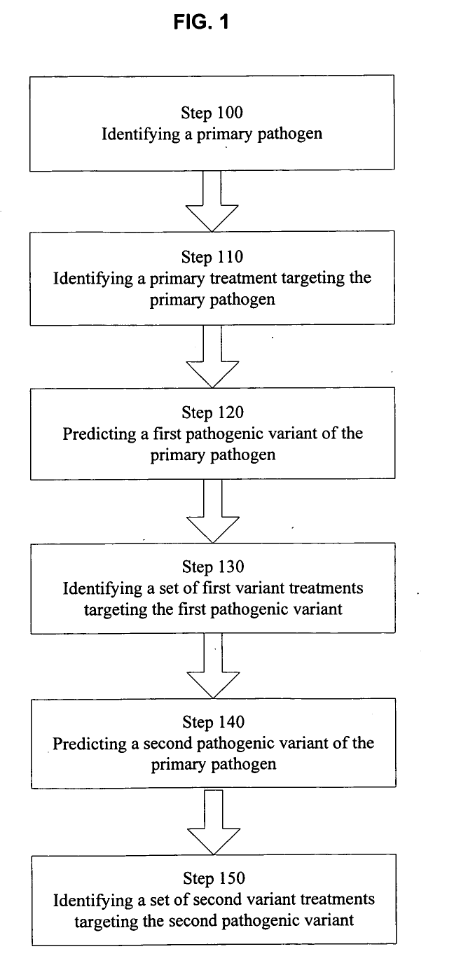

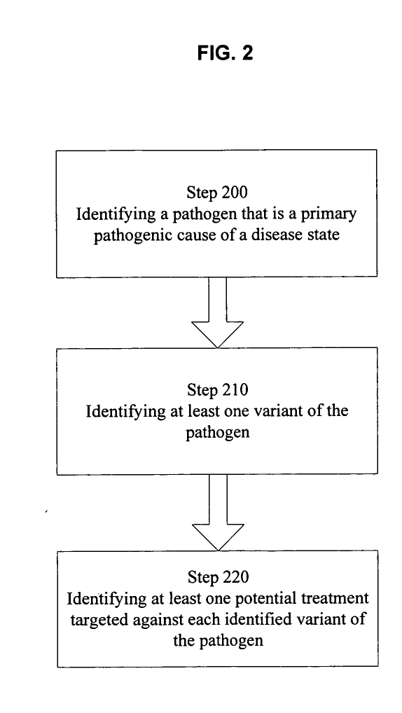

FIG. 1 outlines an embodiment of a method starting at step 100 which includes identifying a primary pathogen. As discussed herein, a primary pathogen is the first identified agent that disrupts the normal metabolic state of an organism. Step 110 describes identifying a primary treatment targeting the primary pathogen. In many instances, step 110 will follow step 100 but this need not always be the case. For example, there may be instances where a treatment is identified that alleviates a disease state before the primary pathogen is identified. Steps 100 and 110 are followed by step 120 which includes predicting a first pathogenic variant of the primary pathogen. Then described in step 130 is identifying a set of first variant treatments targeting the first pathogenic variant. As discussed in reference to steps 100 and 110, step 120 and step 130 may occur in any order or contemporaneously. Step 120 is followed by step 140 which includes predicting a second pathogenic variant of the primary pathogen. Step 150 outlines identifying a set of second variant treatments targeting the second pathogenic variant.

FIG. 2 diagrams a further embodiment of a method. Step 200 includes identifying a pathogen that is a primary pathogenic cause of a disease state. Step 210 comprises identifying at least one variant of the pathogen. Step 220 includes identifying at least one potential treatment targeted against each identified variant of the pathogen. Although the steps outlined in FIG. 2 are shown in a particular order, they need not occur in this order. For example, at least one variant of a pathogen and at least one targeted treatment directed against a variant may be known before the pathogen that is a primary pathogenic cause of a disease state is recognized.

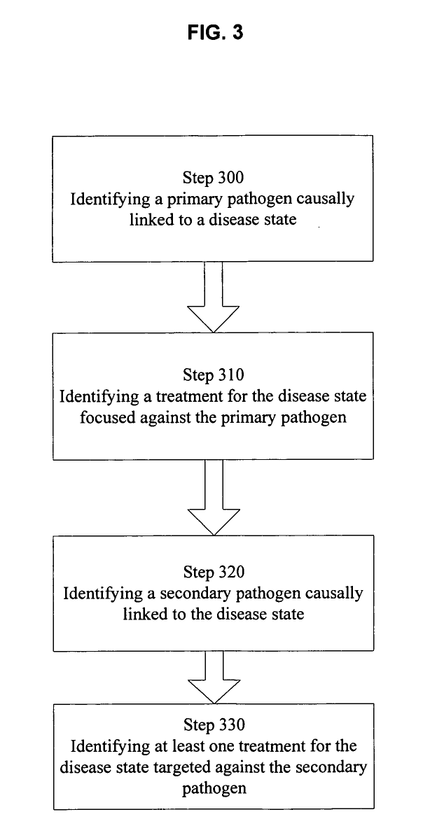

FIG. 3 outlines steps of an embodiment of a method. Step 300 includes identifying a primary pathogen causally linked to a disease state. Outlined in step 310 is identifying a treatment for the disease state focused against the primary pathogen. Step 320 describes identifying a secondary pathogen causally linked to the disease state. Step 330 shows identifying at least one treatment for the disease state targeted against the secondary pathogen. Although the steps in FIG. 3 are shown in sequence, they need not occur in the shown linear order but in some embodiments may occur in an alternate order or be simultaneous.

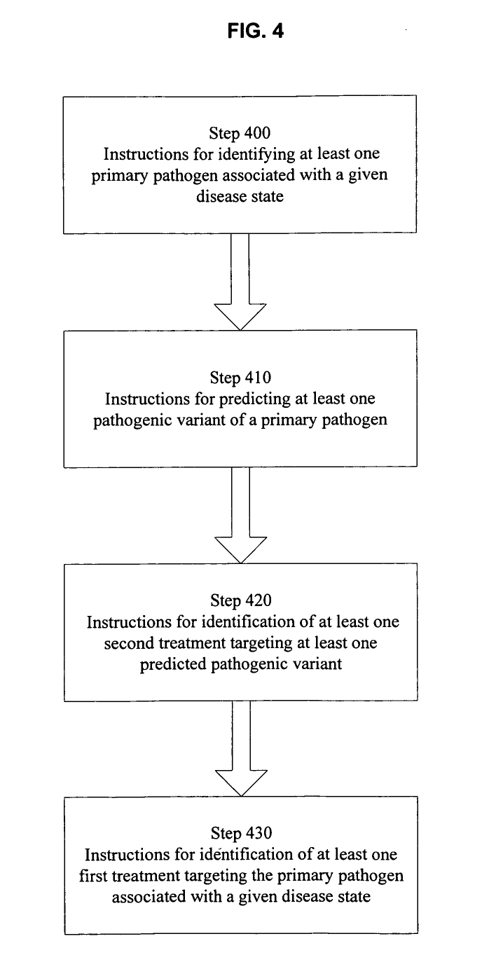

FIG. 4 outlines steps included in a system comprising a computer program for use with a computer system and wherein the computer program includes a plurality of instructions. Step 400 shows instructions for identifying at least one primary pathogen associated with a given disease state. Step 410 includes instructions for predicting at least one pathogenic variant of a primary pathogen. Outlined in step 420 are instructions for identification of at least one second treatment targeting at least one predicted pathogenic variant. Step 430 shows instructions for identification of at least one first treatment targeting the primary pathogen associated with a given disease state. Although steps 420 and 430 are shown in the order listed in this diagram, in some embodiments step 430 may occur before step 420.

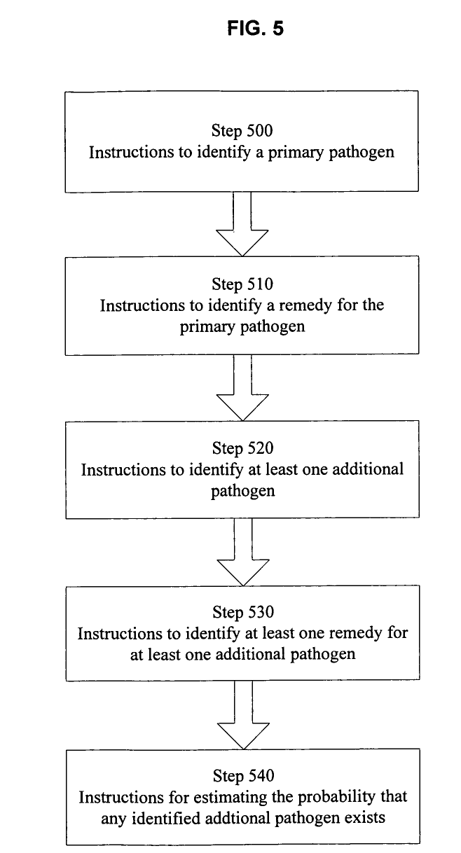

FIG. 5 describes a system comprising a computer readable medium including a computer program for use with a computer system, said computer program including a series of steps. Step 500 shows instructions to identify a primary pathogen. Step 510 describes instructions to identify a remedy for the primary pathogen. Although steps 500 and 510 are shown in the order listed, in some embodiments step 510 may occur before step 500. Outlined in step 520 are instructions to identify at least one additional pathogen. Step 530 includes instructions to identify at least one remedy for at least one additional pathogen. Step 540 shows instructions for estimating the probability that any identified additional pathogen exists. Although steps 520, 530 and 540 are shown in a given order in this diagram, in different embodiments they may occur in any order.

Illustrative Examples of the methods and systems described herein are discussed below.

### EXAMPLE 1

HIV is a pathogen which is the underlying cause of AIDS. HIV has been shown to mutate frequently, with the result that new variants are constantly being produced with varying levels of resistance to commonly employed treatments. For more information regarding the high mutation rate in HIV and its effect on circulating virus, see Coffin, “HIV Population Dynamics in Vivo: Implications for Genetic Variation, Pathogenesis and Therapy” Science 267: 483-489 (1995) which is herein incorporated by reference. Among the HIV genes that frequently mutates is HIV-1 reverse transcriptase (RT), which is essential for HIV replication but is not required for normal cell replication. A group of molecules known as nonnucleoside reverse transcriptase inhibitors (NNRTIs) are known to bind to RT and inhibit its activity. Some of the NNRTIs have been used as treatment for HIV infection.

The molecular structure of RT and at least one NNRTI, Efavirenz, have been described singly and in complex (see Mei et al., “Quantum Study of Mutational Effect in Binding of Efavirenz to HIV-1 RT” Proteins, 59:489-495 (2005), which is herein incorporated by reference). Embodiments of the methods and systems described herein are applicable for the identification of variants of HIV that are most amenable to treatment with NNRTIs in general and Efavirenz in particular, based on specific variants of HIV.

In some circumstances, it may be desirable to initiate therapy for HIV infection in the pre-disease state, or after initial infection but before symptoms corresponding to AIDS. Methods and systems described herein would be applicable to the selection of treatment for HIV infection before a patient's symptoms progressed to AIDS.

In some circumstances, there may be pathogens distinct from HIV which are responsible for AIDS related diseases and methods and systems described herein would also be applicable to those pathogens separately from HIV. For example, infection with cytomegalovirus (CMV) is a significant cause of morbidity and mortality in HIV infected individuals. Variants of CMV with different responses to different treatments have been described, for example see Williams et al., “In Vitro Activities of Benzimidazole D- and L-Ribonucleosides against Herpesviruses” Antiviral Agents and Chemotherapy, 47(7):2186-2192 (2003), which is herein incorporated by reference. Methods and systems described herein are applicable for the identification of CMV variants that are amenable to specific treatments in HIV-infected individuals.

### EXAMPLE 2

Influenza is a disease with significant morbidity as well as mortality worldwide. In many years, the public health burden of influenza is minimal but occasional serious outbreaks occur. Although vaccination for influenza is a key part of public health approaches to managing influenza outbreaks, selection of appropriate vaccination targets is confounded by the rapid emergence of new strains of influenza. The emergence of novel strains, their relative fitness and response to recent vaccines may be predicted. See, for example, a discussion of the genetic and antigenic evolution of influenza A (H3N2) virus in Smith et al., “Mapping the Antigenic and Genetic Evolution of Influenza Virus” Science 305(5682):371-376 (2004), which is herein incorporated by reference. The response of influenza strains carrying specific alterations to treatment with specific antiviral drugs after infection has been described, see for example, Gubareva et al., “Comparison of the Activities of Zanamivir, Oseltamivir and RWJ-270201 Against Clinical Isolates of Influenza Virus and Neuraminidase Inhibitor-Resistant Variants” Antimicrobial Agents and Chemotherapy 45(12):3403 (2001), which is herein incorporated by reference.

Methods and systems described herein are applicable to the identification of circulating influenza strains and their response to treatment. Specific strains and corresponding treatments may be identified. In some embodiments, strains may be predicted based on population-based information regarding recently administered vaccines and/or use of antiviral drugs.

The above referenced technical articles are specifically incorporated herein by reference in their entirety for all that they disclose and teach. In an event of any conflict between the instant application and a referenced technical article, the instant application controls.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All of the above U.S. patents, U.S. patent application publications, U.S. patent applications, foreign patents, foreign patent applications and non-patent publications referred to in this specification and/or listed in any Application Data Sheet, are incorporated herein by reference, in their entireties.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired. The various aspects and embodiments disclosed herein are for purposes of illustration and are not intended to be limiting, with the true scope and spirit being indicated by the following claims.

What is claimed is:

1. A computer-implemented method comprising:
identifying a primary pathogen;
identifying a primary treatment targeting the primary pathogen;
predicting a first pathogenic variant of the primary pathogen;
identifying a set of first variant treatments targeting the first pathogenic variant;
predicting a second pathogenic variant of the primary pathogen; and
identifying a set of second variant treatments targeting the second pathogenic variant.

2. The method as in claim 1, comprising:
selecting at least one identified first variant treatment; and
selecting at least one identified second variant treatment.

3. The method as in claim 2, comprising:
administering the selected first variant treatment and the selected second variant treatment.

4. The method as in claim 2, comprising:
treating a disease state using a combination of treatments, including at least one selected first treatment and at least one selected second treatment.

5. The method as in claim 3, wherein the treatments are administered at the same time.

6. The method as in claim 3, wherein the treatments are administered at different times.

7. The method as in claim 4, wherein the treatments have the same route of delivery into the body of the individual being treated.

8. The method as in claim 4, wherein at least one of the selected treatments may be administered orally, via injection, via intravenous administration, transdermally, nasally, through the lung tissue or via inhalation.

9. The method as in claim 4, wherein at least one of the treatments is administered at a single time.

10. The method as in claim 4 wherein at least one of the treatments comprises a series of treatments carried out over time.

11. The method as in claim 1, wherein the primary pathogen is a virus, a bacterium, a yeast, a mold, a fungus, a mycoplasma, a ureaplasma, a chlamydia, a rickettsia, a nanobacterium, a prion, an agent responsible for a transmissible spongiform encephalopathy (TSE), a multicellular parasite, a protein, an infectious protein, a nucleic acid, a metabolic by-product, a cellular by-product, or a toxin.

12. The method as in claim 1 wherein the primary pathogen is associated with a disease state.

13. The method as in claim 12 wherein the disease state affects a domestic animal.

14. The method as in claim 12 wherein the disease state affects a human.

15. The method as in claim 12 wherein the disease state affects a non-domesticated animal.

16. The method as in claim 1 wherein at least one pathogenic variant is a virus, a bacterium, a yeast, a mold, a fungus, a mycoplasma, a ureaplasma, a chlamydia, a rickettsia, a nanobacterium, a prion, an agent responsible for a transmissible spongiform encephalopathy (TSE), a multicellular parasite, a protein, an infectious protein, a nucleic acid, a metabolic by-product, a cellular by-product, or a toxin.

17. The method as in claim 1 wherein at least one predicted pathogenic variant is a previously identified variant.

18. The method as in claim 1 wherein predicting at least one pathogenic variant is in response to a molecular model of the primary pathogen.

19. The method as in claim 1 wherein predicting at least one first pathogenic variant includes accessing information from a database.

20. The method as in claim 2 wherein selecting at least one treatment includes accessing information from a database.

21. The method as in claim 2 wherein selecting at least one treatment is made in response to a molecular model of a primary pathogen and/or a pathogenic variant.

22-77. (canceled)
